(19) United States
(12) Patent Application Publication
(10) Pub. No.: US 2022/0411374 A1
(43) Pub. Date: Dec. 29, 2022

(54) ALTERNATE PROCESSES FOR THE PREPARATION OF OMECAMTIV MECARBIL

(71) Applicant: DR. REDDY'S LABORATORIES LIMITED, Hyderabad, Telangana (IN)

(72) Inventors: Saikat Sen, West Bengal (IN); Srinivas Oruganti, Hyderabad (IN); Magesh Sampath, Kancheepuram (IN); Azeezulla Baig Mohammed, Hyderabad (IN)

(73) Assignee: DR. REDDY'S LABORATORIES LIMITED, Hyderabad, Telangana (IN)

(21) Appl. No.: 17/767,335

(22) PCT Filed: Oct. 9, 2020
(86) PCT No.: PCT/IB2020/059486

§ 371 Date: Apr. 7, 2022

(30) Foreign Application Priority Data
Oct. 9, 2019 (IN) 201941040736

(51) Int. Cl. C07D 213/75

(52) U.S. Cl. CPC C07D 213/75

(57) ABSTRACT

Aspects of the present application relate to process for the preparation of Omecamtiv mecarbil and salts thereof. Specific aspects relate to novel urea intermediate, preparative process thereof and its use in the preparation of Omecamtiv mecarbil and salts thereof. The improved process for the preparation of Omecamtiv mecarbil are industrially viable.

### INTRODUCTION

Aspects of the present application relate to process for the preparation of Omecamtiv mecarbil and salts thereof. Specific aspects relate to improved processes for Omecamtiv mecarbil and salts thereof.

Omecamtiv mecarbil is a first-in-class cardiac myosin activator, which increases the proportion of myosin heads that are tightly bound to actin and creates a force-producing state that is not associated with cytosolic calcium accumulation. Omecamtiv mecarbil is developed by Cytokinetics Inc. & Licensee, Amgen Inc. and has a chemical name: Methyl 4-[(2-fluoro-3-{[(6-methyl(3-pyridyl))amino]carbonylamino}-phenyl)methyl]piperazine carboxylate and the structure as below.

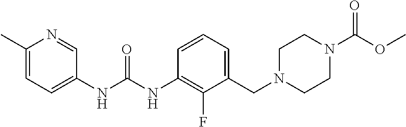

WO 2006009726 A2 (the '726 application) first discloses Omecamtiv mecarbil, its preparative process, pharmaceutical composition and its use in treating heart failure. The process disclosed in the '726 application involves urea formation between piperazinymethyl phenylamine and pyridyl isocyanate, as depicted below.

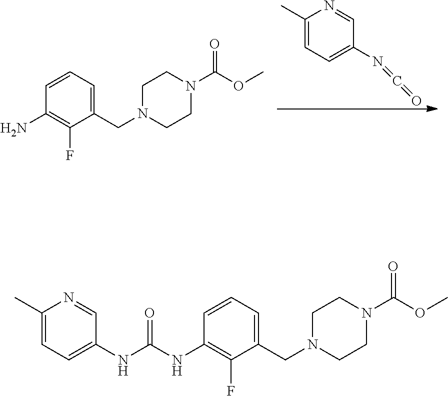

A similar process for the preparation of Omecamtiv mecarbil is disclosed in a subsequent application, WO 2014152270 A1 (the '270 application) which involves the reaction of pyridyl phenylcarbamate with piperazinylmethyl phenylamine, in the presence of alkyl amine, as depicted below. The '270 application further describes that the dihydrochloride of Omecamtiv mecarbil can exist as a crystalline hydrate Form A characterized by an X-ray powder diffraction pattern comprising peaks at about 6.6, 14.9, 20.1, 21.4, and 26.8±0.2° 2θ using Cu Kα radiation and crystalline anhydrous forms B and C, which are metastable.

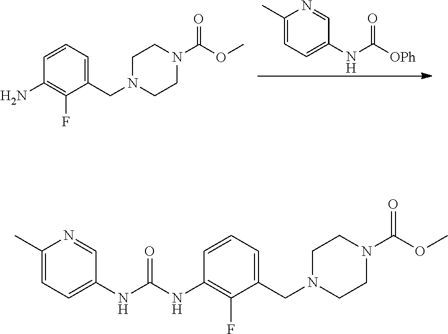

The reported processes suffer from drawbacks such as low yield, sluggish reactions and the like. Also, these procedures involve the use of excess reagents and reactants, ultimately resulting in lack of material balance. Hence, these process are not suitable at commercial scale and there remains a need for an improved process for the preparation of Omecamtiv mecarbil or its salts and reproducible and stable solid forms thereof, which can overcome the disadvantages of the prior art and yet be cost effective and industrially viable.

### SUMMARY

In an aspect, the present application provides a process for the preparation of Omecamtiv mecarbil of formula-I or a salt thereof, comprising the step of reacting a urea compound of formula-IIa, wherein L is hydroxyl or a leaving group selected from the group consisting of halogen and sulfonate, with piperazine compound of formula-III, wherein P is a nitrogen protecting group.

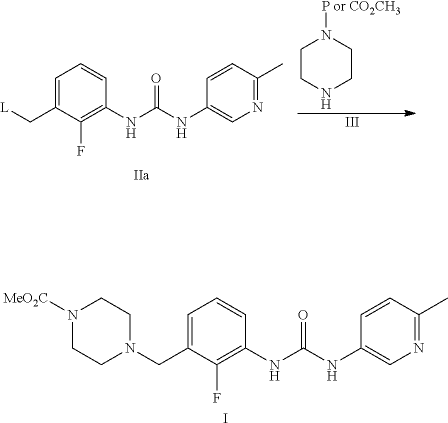

In another aspect, the present application provides a process for the preparation of Omecamtiv mecarbil of formula-I or a salt thereof, comprising the step of reacting a urea compound of formula-IIb with piperazine compound of formula-III, wherein P is a nitro en protecting group.

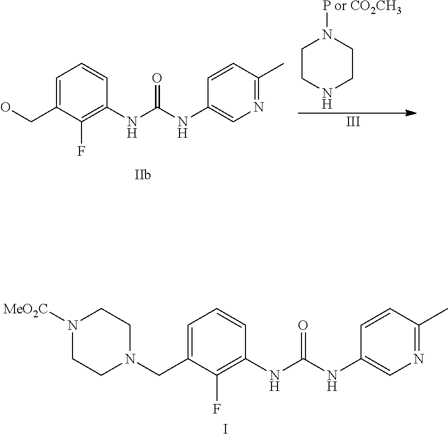

In another aspect, the present application provides a process for the preparation of Omecamtiv mecarbil of formula-I or a salt thereof, comprising the step of reacting a urea compound of formula-IIc with piperazine compound of formula-III, wherein P is a nitrogen protecting group, in the presence of transition metal complex.

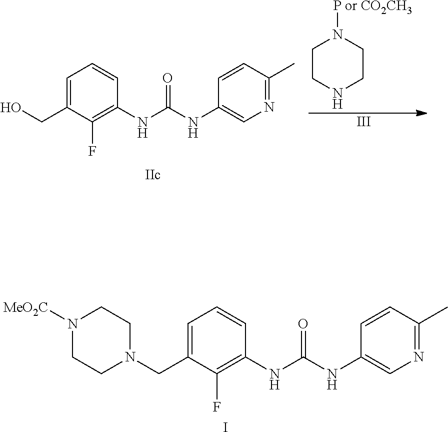

In another aspect, the present application provides a process for the preparation of Omecamtiv mecarbil of formula-I or a salt thereof, comprising the step of reacting a urea compound of formula-IId, wherein R is a group selected from hydroxy, halogen, alkoxy, aryloxy, oxycarbonylalkyl, and oxycarbonylaryl, with piperazine compound of formula-III, wherein P is a nitrogen protecting group, optionally through the isolation of the amide intermediate.

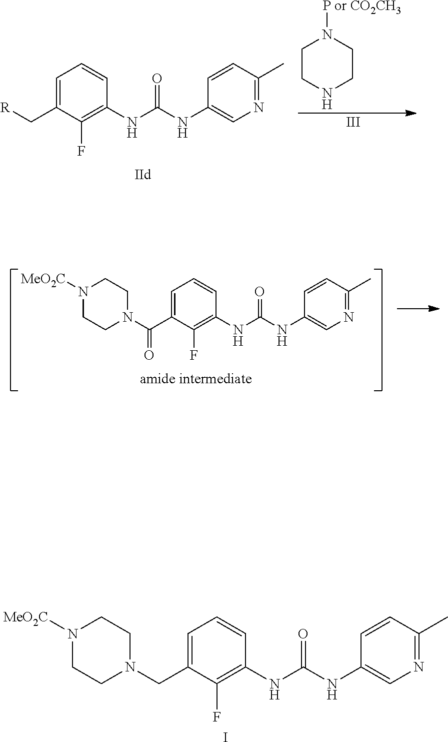

In another aspect, the present application provides a process for the preparation of urea compound of formula-II, comprising the step of reacting an aniline compound of formula-IV, wherein R₂ is selected from the group consisting of cyano, formyl, carboxylic acid or ester thereof, hydroxymethyl, sulfonyloxy methyl and halomethyl, with 5-isocyanato-2-methylpyridine.

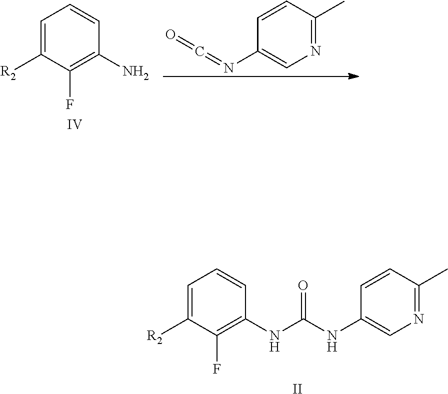

In another aspect, the present application provides a process for the preparation of urea compound of formula-II, comprising the step of reacting an aniline compound of formula-IV, wherein R₂ is selected from the group consisting of cyano, formyl, carboxylic acid or ester thereof, hydroxymethyl, sulfonyloxy methyl and halomethyl, with 6-methylpyridin-3-amine in the presence of a carbonyl source.

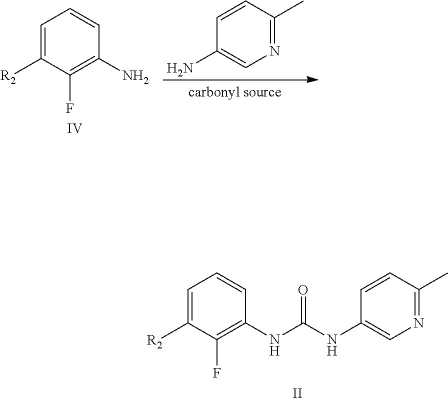

In another aspect, the present application provides a process for the preparation of urea compound of formula-II, comprising the step of reacting a aniline compound of formula-IV, wherein R₂ is selected from the group consisting of cyano, formyl, carboxylic acid or ester thereof, hydroxymethyl, sulfonyloxy methyl and halomethyl with a derivatized 6-methylpyridin-3-amine compound of formula V, wherein R₃ is selected from the group consisting of halogen, alkoxy, aryloxy and heteroaryl.

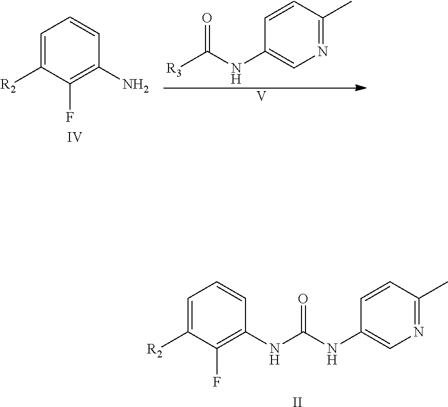

In another aspect, the present application provides a process for the preparation of urea compound of formula-II, comprising the step of reacting a derivatized aniline compound of formula-IVa, wherein R₂ is selected from the group consisting of cyano, formyl, carboxylic acid or ester thereof, hydroxymethyl, sulfonyloxy methyl and halomethyl; R₃ is selected from the group consisting of halogen, alkoxy, aryloxy and heteroaryl, with 6-methylpyridin-3-amine.

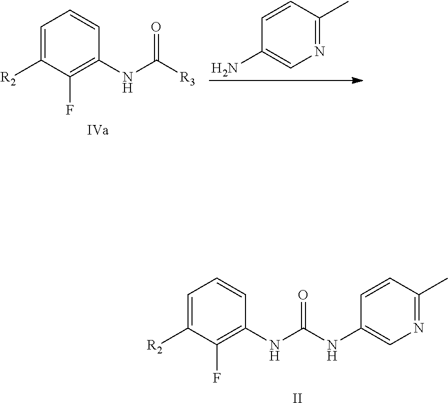

In another aspect, the present application provides a process for the preparation of urea compound of formula-II, comprising the step of reacting an isocyanate of formula-IVb, wherein R₂ is selected from the group consisting of cyano, formyl, carboxylic acid or ester thereof, hydroxymethyl, sulfonyloxy methyl and halomethyl, with 6-methylpyridin-3-amine.

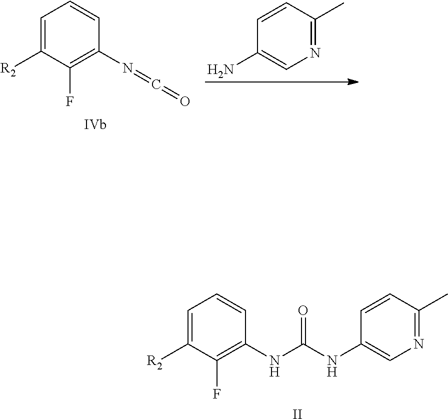

In another aspect, the present application provides a urea compound of formula-II, wherein R₂ is selected from the group consisting of cyano, formyl, carboxylic acid or ester thereof, hydroxymethyl, sulfonyloxy methyl and halomethyl.

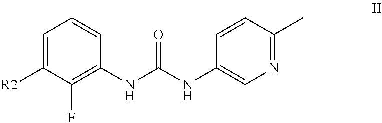

In another aspect, the present application provides a process for the preparation of Omecamtiv mecarbil or a salt thereof, comprising a step of converting urea compound of formula-II to Omecamtiv mecarbil, wherein R₂ is selected from the group consisting of cyano, formyl, carboxylic acid or ester thereof, hydroxymethyl, sulfonyloxy methyl and halomethyl.

In another aspect, the present application provides a process for the preparation of Omecamtiv mecarbil or a salt thereof, comprising the step of reacting a derivatized aniline of formula VI, wherein R₃ is selected from the group consisting of halogen, alkoxy, aryloxy and heteroaryl, with 6-methylpyridin-3-amine in the presence of a base.

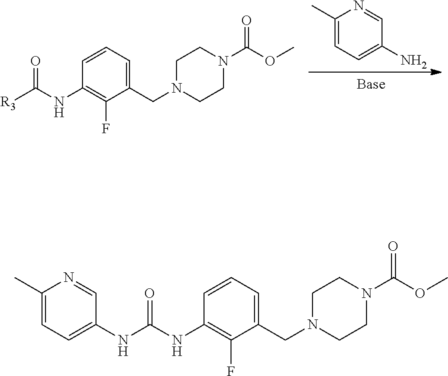

In another aspect, the present application provides a process for the preparation of Omecamtiv mecarbil or a salt thereof, comprising the step of reacting methyl 4-(3-amino-2-fluorobenzyl)piperazine-1-carboxylate with 6-methylpyridin-3-amine in the presence of a carbonyl source.

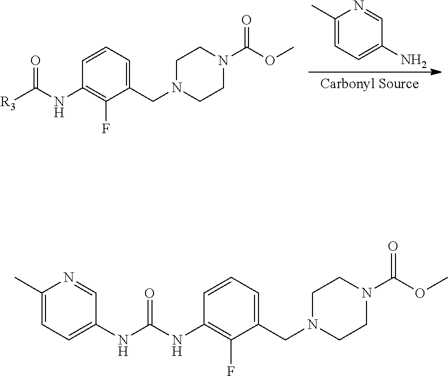

In another aspect, the present application provides a process for the preparation of Omecamtiv mecarbil or a salt thereof, comprising the step of reacting methyl 4-(2-fluoro-3-isocyanatobenzyl)piperazine-1-carboxylate with 6-methylpyridin-3-amine.

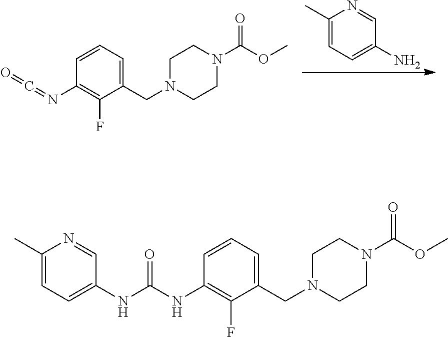

In another aspect, the present application provides a process for the preparation of Omecamtiv mecarbil or a salt thereof, comprising the step of reacting methyl 4-(3-cyano-2-fluorobenzyl)piperazine-1-carboxylate with 6-methylpyridin-3-amine in the presence of Trifluoroacetic anhydride (TFAA)/Dimethylsulfoxide (DMSO).

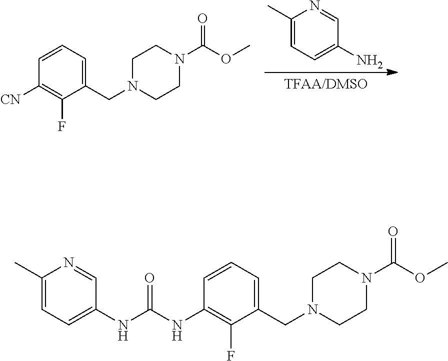

### BRIEF DESCRIPTION OF THE DRAWING

FIG. 1 is an illustrative X-ray powder diffraction pattern of Omecamtiv mecarbil free base prepared by the method of Example No 12.

FIG. 2 is an illustrative X-ray powder diffraction pattern of Omecamtiv mecarbil free base prepared by the method of Example No 7.

### DETAILED DESCRIPTION

In an aspect, the present application provides a process for the preparation of Omecamtiv mecarbil of formula-I or a salt thereof, comprising the step of reacting a urea compound of formula-IIa, wherein L is hydroxy or a leaving group selected from the group consisting of halogen and sulfonate, with piperazine compound of formula-III, wherein P is a nitrogen protecting group.

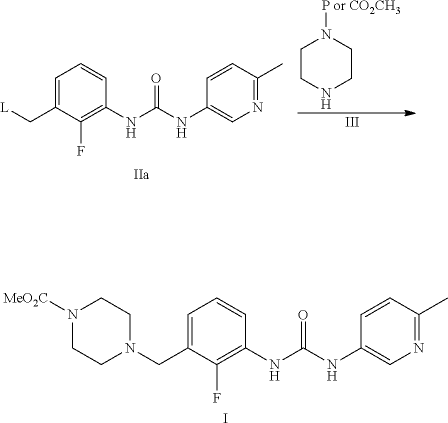

In embodiments, the process of this aspect may be carried out in the presence of a base and an organic solvent. Base may be include, but not limited to alkali metal carbonates, such as, for example, sodium carbonate, potassium carbonate, lithium carbonate, cesium carbonate, or the like; alkali metal bicarbonates, such as, for example, sodium bicarbonate, potassium bicarbonate, or the like; organic bases, such as triethylamine, diisopropylethylamine, 1,8-Diazabicyclo[5.4.0]undec-7-ene (DBU), pyridine, 2,6-dimethylpyridine, N,N-dimethylaminopyridine or the like.

In embodiments, the process of this aspect may be carried out in the presence of catalyst such as alkali metal halides, such as, potassium iodide, sodium iodide or the like; phase transfer reagents, such as, tetrabutylammonium bromide, triethylbenzylammonium chloride, tributylphosphonium bromide, 18-crown-6 or the like.

In embodiments, L is a halogen selected from the group consisting of chlorine, bromine, iodine and fluorine. In alternate embodiments, L is sulfonate selected from the group consisting of methyl sulfonate, ethyl sulfonate, p-toluene sulfonate, benzene sulfonate, trifluoromethanesulfonate or the like.

In embodiments, when L is hydroxy, the hydroxy may be converted to corresponding sulfonate or halogen, according to the methods known in the art or according to the procedure described in the instant specification, and then the corresponding sulfonate or halide compound may be reacted with piperazine compound of formula-III to obtain Omecamtiv mecarbil. In embodiments, the sulfonate or halide compounds may be isolated or reacted in situ.

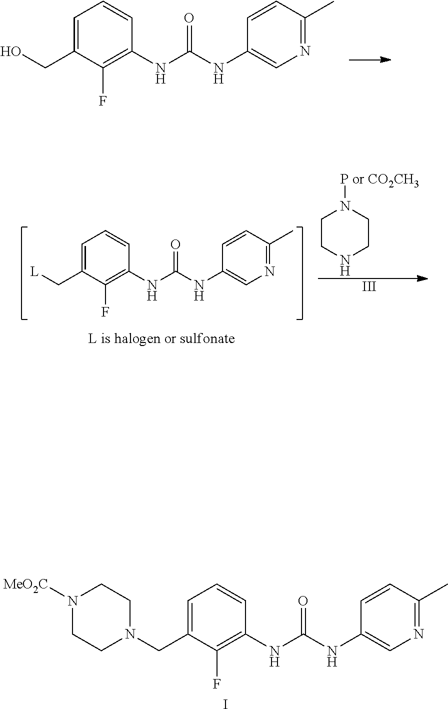

In embodiments, P is suitable nitrogen protecting group obtained using suitable protecting reagents including but not limited to alkyl halides such as benzyl bromide, 4-methoxybenzyl bromide, 2,4-dimethoxybenzyl bromide, triphenylmethyl chloride, methyl iodide, allyl bromide and the likes thereof; oxycarbonyl chlorides or anhydrides such as Fluorenylmethyloxycarbonyl chloride, benzyloxycarbonyl chloride, Methyl chloroformate, ethyl chloroformate, di-tert-butyl dicarbonate and the likes thereof, acid chloride such as acetyl chloride, benzoyl chloride, trifluoroacetyl chloride and the likes thereof; sulfonic acid chlorides such as methyl sulfonyl chloride, ethyl sulfonylchloride, p-toluene sulfonylchloride and the likes thereof.

In embodiments, the protecting group may be removed after the reaction of urea compound IIa with piperazine compound III.

Organic solvents that may be used in the process of this aspect include, but not limited to polar aprotic solvents such as dimethyl formamide, dimethyl sulfoxide, dimethyl acetamide, acetonitrile and the likes thereof; halogenated hydrocarbon solvents such as dichloromethane, ethylene dichloride, chloroform, or the like thereof, aliphatic hydrocarbon solvent such as pentane, hexane, heptane, cyclohexane and the likes thereof; aromatic hydrocarbon solvent such as benzene, toluene, xylene and the likes thereof, ether solvents such as methyl t-butyl ether, diethylether, tetrahydrofuran and the likes thereof, ester solvent such as ethyl acetate, isopropyl acetate and the likes thereof.

In embodiments, the reaction of this aspect may be carried out at a suitable temperature and for time sufficient for the formation of Omecamtive mecarbil, at about 0° C. or above and for at least 1 hour or longer.

In another aspect, the present application provides a process for the preparation of Omecamtiv mecarbil of formula-I or a salt thereof, comprising the step of reacting a urea compound of formula-IIb with piperazine compound of formula-III, wherein P is a nitrogen protecting group.

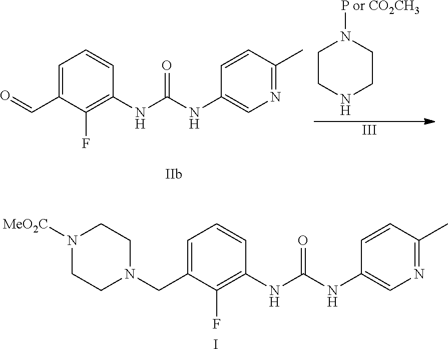

In embodiments, the process of this aspect may be carried out under suitable reductive amination conditions known in the art. In embodiments, the reductive amination may be carried out in the presence of a suitable reducing agent and an organic solvent.

Reducing agent may include but not limited to sodium borohydride, sodium cyanoborohydride, sodium triacetoxyborohydride, decaborane, BH3-pyridine complex, trichlorosilyl hydride, trimethylsilyl hydride, phenylsilane, polymethylhrdrosiloxane, lithium borohydride, zinc borohydride, and hydrogen in presence of Pd, Pt, Ni or Ru.

In embodiments, the reaction between urea compound of formula-IIb and piperazine compound of formula-III may be carried out in the presence of metal triflates and an organic solvent. Metal triflates that can be used include, but not limited to iron triflate, Scandium triflate, Aluminium triflate, zinc triflate, copper triflate, manganese triflate, titanium triflate, gallium triflate and the likes thereof. Organic solvent may be selected from the group consisting of methanol, isopropyl alcohol, ethyl acetate, dichloromethane, dichloroethane, chloroform, tetrahydrofuran, 2-methyl tetrahydrofuran, diethyl ether, ethyl acetate, isopropyl acetate and the likes thereof.

In embodiments, the reaction of this aspect may be carried out at a suitable temperature and for time sufficient for the formation of Omecamtive mecarbil, at about 0° C. or above and for at least 1 hour or longer.

In another aspect, the present application provides a process for the preparation of Omecamtiv mecarbil of formula-I or a salt thereof, comprising the step of reacting a urea compound of formula-IIc with Piperazine compound of formula-III, wherein P is a nitrogen protecting group, in the presence of transition metal complex.

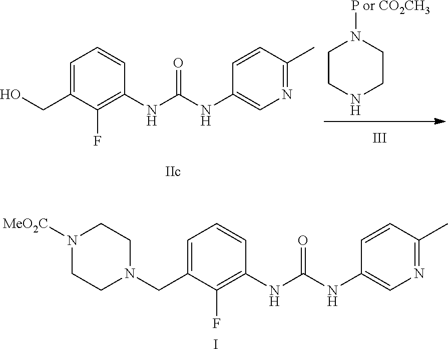

In embodiments, compound of formula IIc is converted into its carbonyl derivative IIb by the removal of hydrogen with a transition metal complex. The carbonyl derivative being reactive than the precursor alcohol, readily reacts in situ with piperazine of formula III to give corresponding imine intermediate. Thereafter transition metal complex returns the borrowed hydrogen, leading to reduction of imine intermediate to Omecamtiv mecarbil

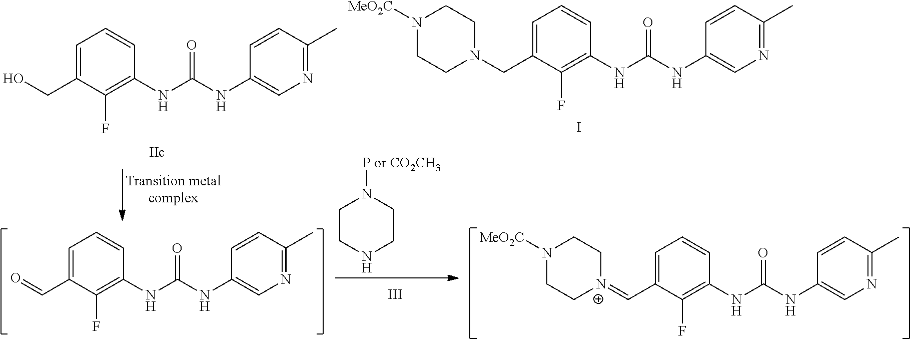

Transition metal complex, that can be used may include, but not limited to RuCl₂(PPh₃)₃, [Ru(p-cymene)Cl2]2, [Cp*IrCl2]2, Ru3(CO)12, Ru(codxcot), CpRuCl(PPh3)2, [Ir(cod)Cl]2, RhCl2(PPh3)₃, IrCl₂(PPh₃)₃, PdCl2, Pd(OAc)₂, 5% Pd/C, Pd(dba)₂, NiCl₂, Cu₂O, other complexes with transition metals Ru, Rd, Ir, Au and the like.

Organic solvent may be selected from the group consisting of aliphatic hydrocarbon solvent such as pentane, hexane, heptane, cyclohexane and the likes thereof; aromatic hydrocarbon solvent such as benzene, toluene, xylene and the likes thereof, ester solvent such as, ethyl acetate, isopropyl acetate and the likes thereof, chlorinated solvents such as, dichloromethane dichloroethane, chloroform and the likes thereof, ether solvents such as tetrahydrofuran, 2-methyl tetrahydrofuran, diethylether, diisopropyl ether, MTBE, dioxane, and dimethoxyethane and the likes thereof.

In embodiments, the reaction of this aspect may be carried out at a suitable temperature and for time sufficient for the formation of Omecamtive mecarbil, at about 0° C. or above and for at least 1 hour or longer.

In another aspect, the present application provides a process for the preparation of Omecamtiv mecarbil of formula-I or a salt thereof, comprising the step of reacting a urea compound of formula-IId, wherein R is a group selected from hydroxy, halogen, alkoxy, aryloxy, oxycarbonylalkyl, and oxycarbonylaryl, with piperazine compound of formula-III, wherein P is a nitrogen protecting group, optionally through the isolation of amide intermediate.

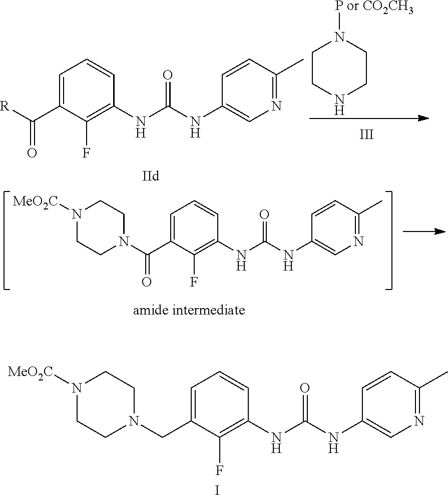

In embodiments, the process of this aspect may be carried out under suitable acylation conditions known in the art to obtain corresponding amide intermediate followed by its reduction using suitable reducing agent to obtain Omecamtiv mecarbil. In embodiments, the amide intermediate may be isolated before reduction or may be reduced in situ.

In embodiments, the urea compound of formula IId may be reacted with piperazine compound of formula III, either in the form of a carboxylic acid, when R is hydroxy group or in the form of a reactive derivative such as carboxylic acid halide, ester and anhydride, when R is other than hydroxyl group.

In embodiments, the acylation may be carried out in the presence of base and a suitable solvent. Base may include, but not limited to alkali metal carbonates, such as, for example, sodium carbonate, potassium carbonate, lithium carbonate, cesium carbonate, or the like; alkali metal bicarbonates, such as, for example, sodium bicarbonate, potassium bicarbonate, or the like; organic base, such as triethylamine, diisopropylethylamine, 1,8-Diazabicyclo[5.4.0]undec-7-ene (DBU), pyridine, 2,6-dimethylpyridine, N,N-dimethylaminopyridine or the like; metal alkoxides, such as, for example, sodium tert-butoxide, lithium tert-butoxide, potassium tert-butoxide, sodium methoxide, sodium ethoxide or the like.

In embodiments, the acylation may be carried out at a suitable temperature and for time sufficient for the formation of amide intermediate, at about 0° C. or above and for at least 1 hour or longer.

In embodiments, the reduction of amide intermediates may be carried out in the presence of suitable reducing agent. In embodiments, the reduction of amide intermediates may be carried under suitable metal catalyzed reduction conditions. Reducing agent may include, but not limited to LiAlH₄, H₂PtCl₆/1,1,3,3-tetramethyldisiloxane, H₂PtCl₆/1,2-bis(dimethylsilyl)benzene, NiCl₂(dme)/PhSiH₃, Lithium Borohydride, Sodium Borohydride (NaBH₄), BH₃-THF complex, Sodium triacetoxyborohydride, sodium cyanoborohydride, or the like.

In embodiments, the reduction of amide intermediate may be carried out at a suitable temperature and for time sufficient for the formation of Omecamtiv mecarbil, at about 0° C. or above and for at least 1 hour or longer.

In embodiments, the reduction of amide intermediate may be carried out in the presence of a suitable solvent. Examples of such solvents include, but are not limited to: ethers, such as diethyl ether, diisopropyl ether, tetrahydrofuran, MTBE, dioxane, and dimethoxyethane; halogenated hydrocarbons, such as dichloromethane, chloroform, carbon tetrachloride, and chlorobenzene; aromatic hydrocarbons, such as toluene, benzene, xylene and the likes thereof.

In another aspect, the present application provides a process for the preparation of urea compound of formula-II, comprising the step of reacting an aniline compound of formula-IV, wherein R₂ is selected from the group consisting of cyano, formyl, carboxylic acid or ester thereof, hydroxymethyl, sulfonyloxy methyl and halomethyl, with methylpyridyl isocyanate.

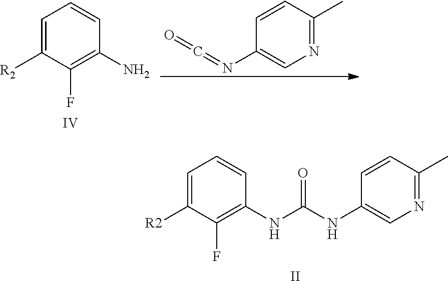

In embodiments, the process of present aspect may be carried out may be carried out in the presence of a suitable solvent. Solvent may be selected from the group consisting of ethers, such as diethyl ether, diisopropyl ether, tetrahydrofuran, MTBE, dioxane, and dimethoxyethane; halogenated hydrocarbons, such as dichloromethane, dichloroethane chloroform, carbon tetrachloride, and chlorobenzene, 1,2-dichlorobenzene; aromatic hydrocarbons, such as toluene, benzene, xylene; aliphatic hydrocarbons such as pentane, hexane, heptane; nitriles such as acetonitrile, esters such as ethyl acetate, isopropyl acetate and polar aprotic solvents such as DMF, DMSO, DMAc, any mixtures of two or more thereof.

In embodiments, the process may be carried out at a suitable temperature and for time sufficient for the formation of urea compound of formula-II, at about 0° C. or above and for at least 1 hour or longer.

In another aspect, the present application provides a process for the preparation of urea compound of formula-II, comprising the step of reacting an aniline compound of formula-IV, wherein R₂ is selected from the group consisting of cyano, formyl, carboxylic acid or ester thereof, hydroxylmethyl, sulfonyloxy methyl and halomethyl, with 6-methylpyridin-3-amine in the presence of a carbonyl source.

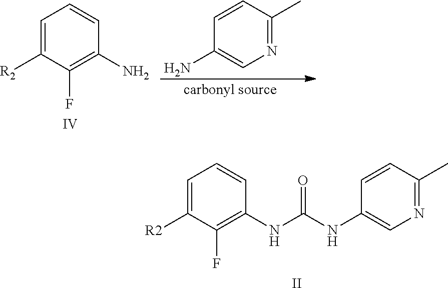

In embodiments, the process of present aspect may be carried out in the presence of a suitable carbonyl source such as carbon monoxide; phosgene; phosgene equivalents such as diphosgene and triphosgene, carbonyldiimidazole (CDI) and 1,1-carbonylbisbenzotriazole; dialkyl carbonates such as, dimethyl carbonate diethyl carbonate, diisopropyl carbonate, S,S-Dimethyldithiocarbonate (DMDTC), di-tert-butyl dicarbonate and bis(4-nitrophenyl)carbonate; alkyl or aryl carbodiimides such as diisopropyl carbodimide and dicyclohexyl carbodiimide; alkyl or aryl chloroformates such as phenyl chloroformate and the likes thereof.

In embodiments, the process may be carried out may be carried out in the presence of a suitable solvent. Solvent may be selected from the group consisting of ethers, such as diethyl ether, diisopropyl ether, tetrahydrofuran, MTBE, dioxane, and dimethoxyethane; halogenated hydrocarbons, such as dichloromethane, dichloroethane, chloroform, carbon tetrachloride, and chlorobenzene, 1,2-dichlorobenzene; aromatic hydrocarbons, such as toluene, benzene, xylene; aliphatic hydrocarbons such as pentane, hexane, heptane, nitriles such as acetonitrile, esters such as ethyl acetate, isopropyl acetate and polar aprotic solvents such as DMF, DMSO, DMAc, any mixtures of two or more thereof.

In embodiments, the process may be carried out in the presence of a base. Base may be selected from alkali metal carbonates, such as, for example, sodium carbonate, potassium carbonate, lithium carbonate, cesium carbonate, or the like; alkali metal bicarbonates, such as, for example, sodium bicarbonate, potassium bicarbonate, or the like; organic base, such as triethylamine, diisopropylethylamine, 1,8-Diazabicyclo[5.4.0]undec-7-ene (DBU), pyridine, 2,6-dimethylpyridine, N,N-dimethylaminopyridine or the like.

In embodiments, the process may be carried out at a suitable temperature and for time sufficient for the formation of urea compound of formula-II, at about 0° C. or above and for at least 1 hour or longer.

In another aspect, the present application provides a process for the preparation of urea compound of formula-II, comprising the step of reacting a aniline compound of formula-IV, wherein R₂ is selected from the group consisting of cyano, formyl, carboxylic acid or ester thereof, hydroxymethyl, sulfonyloxy methyl and halomethyl with a derivatized 6-methylpyridin-3-amine compound of formula V, wherein R₃ is selected from the group consisting of halogen, alkoxy, aryloxy and heteroaryl.

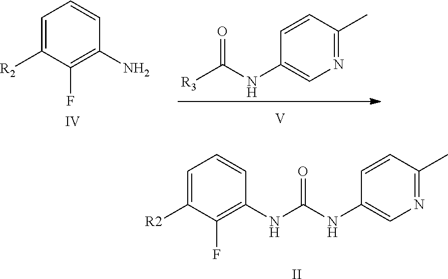

In embodiments, the process of this aspect may be carried out in the presence of a base and an organic solvent. Base may include, but not limited to organic base such as triethylamine, diisopropylethylamine, 1,8-Diazabicyclo[5.4.0]undec-7-ene (DBU), pyridine, 2,6-dimethylpyridine, N,N-dimethylaminopyridine, or the like; as metal carbonates such as sodium carbonate, potassium carbonate, lithium carbonate, cesium carbonate, or the like; metal bicarbonates such as, sodium bicarbonate, potassium bicarbonate, or the like.

Solvent may be selected from the group consisting of ethers, such as diethyl ether, diisopropyl ether, tetrahydrofuran, MTBE, dioxane, and dimethoxyethane; halogenated hydrocarbons, such as dichloromethane, dichloroethane, chloroform, carbon tetrachloride, and chlorobenzene, 1,2-dichlorobenzene; aromatic hydrocarbons, such as benzene, toluene, xylene or the like; aliphatic hydrocarbons such as pentane, hexane and heptane or the like, nitriles such as acetonitrile, esters such as ethylacetate and polar aprotic solvents such as DMF, DMSO, DMAc, any mixtures of two or more thereof.

In embodiments, the process may be carried out at a suitable temperature and for time sufficient for the formation of urea compound of formula-II, at about 0° C. or above and for at least 1 hour or longer.

In another aspect, the present application provides a process for the preparation of urea compound of formula-II, comprising the step of reacting a aniline compound of formula-IVa, wherein R₂ is selected from the group consisting of cyano, formyl, carboxylic acid or ester thereof, sulfonyloxy methyl and halomethyl; R₃ is selected from the group consisting of halogen, alkoxy, aryloxy and heteroaryl, with 6-methylpyridin-3-amine.

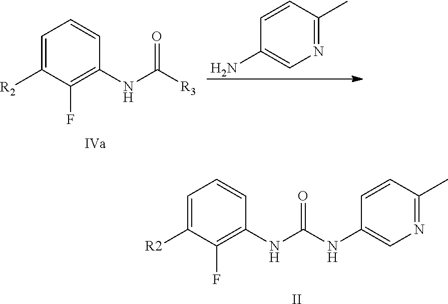

In embodiments, the process of this aspect may be carried out in the presence of a base and an organic solvent. Base may include, but not limited to organic base such as triethylamine, diisopropylethylamine, 1,8-Diazabicyclo[5.4.0]undec-7-ene (DBU), pyridine, 2,6-dimethylpyridine, N,N-dimethylaminopyridine, or the like; or inorganic base such as metal carbonates such as sodium carbonate, potassium carbonate, lithium carbonate, cesium carbonate, or the like; metal bicarbonates such as, sodium bicarbonate, potassium bicarbonate, or the like.

Solvent may be selected from the group consisting of ethers, such as diethyl ether, diisopropyl ether, tetrahydrofuran, MTBE, dioxane, and dimethoxyethane; halogenated hydrocarbons, such as dichloromethane, dichloroethane, chloroform, carbon tetrachloride, and chlorobenzene, 1,2-dichlorobenzene; aromatic hydrocarbons, such as benzene, toluene, xylene or the like; aliphatic hydrocarbons such as pentane, hexane and heptane or the like, nitriles such as acetonitrile, esters such as ethylacetate and polar aprotic solvents such as DMF, DMSO, DMAc, any mixtures of two or more thereof.

In embodiments, the process may be carried out at a suitable temperature and for time sufficient for the formation of urea compound of formula-II, at about 0° C. or above and for at least 1 hour or longer.

In another aspect, the present application provides a process for the preparation of urea compound of formula-II, comprising the step of reacting an isocyanate of formula-IVb, wherein R₂ is selected from the group consisting of cyano, formyl, carboxylic acid or ester thereof, hydroxymethyl, sulfonyloxy methyl and halomethyl, with 6-methylpyridin-3-amine.

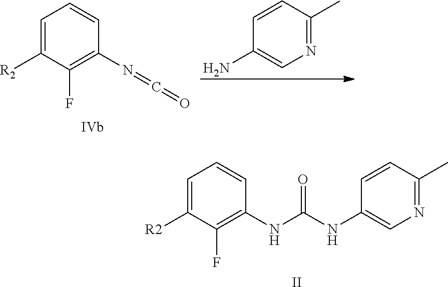

In embodiments, the process of present aspect may be carried out may be carried out in the presence of a suitable solvent. Solvent may be selected from the group consisting of ethers, such as diethyl ether, diisopropyl ether, tetrahydrofuran, MTBE, dioxane, and dimethoxyethane; halogenated hydrocarbons, such as dichloromethane, dichloroethane chloroform, carbon tetrachloride, and chlorobenzene, 1,2-dichlorobenzene; aromatic hydrocarbons, such as toluene, benzene, xylene; aliphatic hydrocarbons such as pentane, hexane, heptane; nitriles such as acetonitrile, esters such as ethyl acetate, isopropyl acetate and polar aprotic solvents such as DMF, DMSO, DMAc, any mixtures of two or more thereof.

In embodiments, the process may be carried out at a suitable temperature and for time sufficient for the formation of urea compound of formula-II, at about 0° C. or above and for at least 1 hour or longer.

In another aspect, the present application provides a urea compound of formula-II, wherein R₂ is selected from the group consisting of cyano, formyl, carboxylic acid or ester thereof, sulfonyloxy methyl and halomethyl.

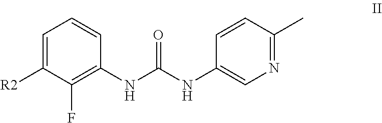

In another aspect, the present application provides a process for the preparation of Omecamtiv mecarbil or salts thereof through the formation of the urea compound of formula-II. In embodiments, the urea compound of formula-II may be converted to Omecamtiv mecarbil according to any of the process described in the present application.

In another aspect, the present application provides a process for the preparation of Omecamtiv mecarbil or a salt thereof, comprising the step of converting a urea compound of formula-II to Omecamtiv mecarbil, wherein R₂ is selected from the group consisting of cyano, formyl, carboxylic acid or ester thereof, hydroxymethyl, sulfonyloxy methyl and halomethyl. In embodiments, the process comprises conversion of urea compound of formula II by reacting with piperazine compound of formula-III. In embodiments, urea compound of formula II may be reacted with piperazine compound of formula-III according to any of the aspects of the instant application.

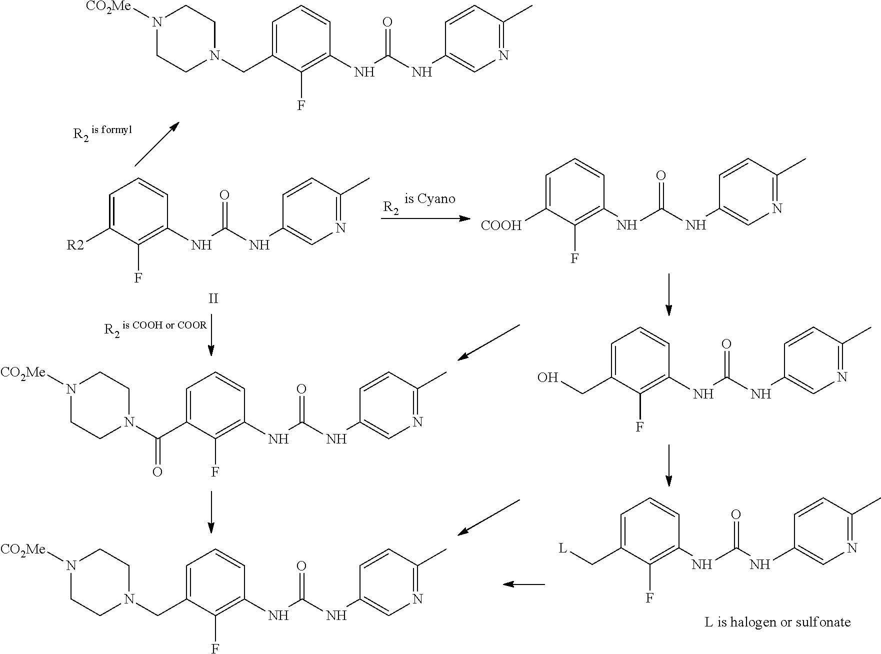

In another aspect, the present application provides a process for the preparation of Omecamtiv mecarbil or a salt thereof, comprising the step of reacting methyl 4-(3-amino-2-fluorobenzyl)piperazine-1-carboxylate with 6-methylpyridin-3-amine in the presence of a carbonyl source.

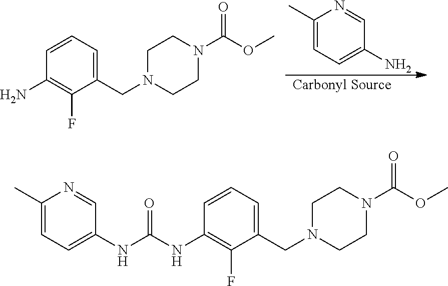

In embodiments, the process of present aspect may be carried out in the presence of a suitable carbonyl source such as carbon monoxide; phosgene; phosgene equivalents such as diphosgene and triphosgene, carbonyldiimidazole (CDI) and 1,1-carbonylbisbenzotriazole; dialkyl carbonates such as, dimethyl carbonate diethyl carbonate, diisopropyl carbonate, S,S-Dimethyldithiocarbonate (DMDTC), di-tert-butyl dicarbonate and bis(4-nitrophenyl)carbonate; alkyl or aryl carbodiimides such as diisopropyl carbodimide and dicyclohexyl carbodiimide; alkyl or aryl chloroformates such as phenyl chloroformate and the likes thereof.

In embodiments, the process may be carried out in the presence of a suitable solvent. Solvent may be selected from the group consisting of ethers, such as diethyl ether, diisopropyl ether, tetrahydrofuran, MTBE, dioxane, and dimethoxyethane; halogenated hydrocarbons, such as dichloromethane, dichloroethane, chloroform, carbon tetrachloride, and chlorobenzene, 1,2-dichlorobenzene; aromatic hydrocarbons, such as toluene, benzene, xylene; aliphatic hydrocarbons such as pentane, hexane, heptane, nitriles such as acetonitrile, esters such as ethyl acetate, isopropyl acetate and polar aprotic solvents such as DMF, DMSO, DMAc, any mixtures of two or more thereof.

In embodiments, the process may be carried out in the presence of a base. Base may be selected from alkali metal carbonates, such as, for example, sodium carbonate, potassium carbonate, lithium carbonate, cesium carbonate, or the like; alkali metal bicarbonates, such as, for example, sodium bicarbonate, potassium bicarbonate, or the like; organic base, such as triethylamine, diisopropylethylamine, 1,8-Diazabicyclo[5.4.0]undec-7-ene (DBU), pyridine, 2,6-dimethylpyridine, N,N-dimethylaminopyridine or the like.

In embodiments, the process may be carried out at a suitable temperature and for time sufficient for the formation of urea compound of formula-II, at about 0° C. or above and for at least 1 hour or longer.

In another aspect, the present application provides a process for the preparation of Omecamtiv mecarbil or a salt thereof, comprising the step of reacting derivatized aniline of formula VI, wherein R₃ is selected from the group consisting of halogen, alkoxy, aryloxy and heteroaryl, with 6-methylpyridin-3-amine in the presence of a base.

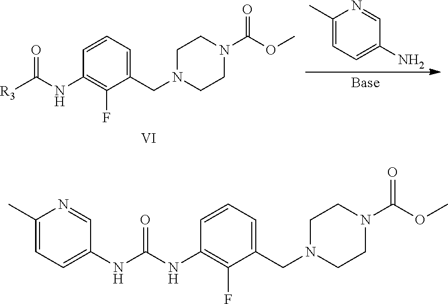

In embodiments, the process may be carried out in the presence of a suitable solvent. Solvent may be selected from the group consisting of ethers, such as diethyl ether, diisopropyl ether, tetrahydrofuran, MTBE, dioxane, and dimethoxyethane; halogenated hydrocarbons, such as dichloromethane, dichloroethane, chloroform, carbon tetrachloride, and chlorobenzene, 1,2-dichlorobenzene; aromatic hydrocarbons, such as toluene, benzene, xylene; aliphatic hydrocarbons such as pentane, hexane, heptane, nitriles such as acetonitrile, esters such as ethyl acetate, isopropyl acetate and polar aprotic solvents such as DMF, DMSO, DMAc, any mixtures of two or more thereof.

In embodiments, the process may be carried out in the presence of a base. Base may be selected from alkali metal carbonates, such as sodium carbonate, potassium carbonate, lithium carbonate, cesium carbonate, or the like; alkali metal bicarbonates, such as sodium bicarbonate, potassium bicarbonate, or the like; organic base, such as triethylamine, diisopropylethylamine, 1,8-Diazabicyclo[5.4.0]undec-7-ene (DBU), pyridine, 2,6-dimethylpyridine, N,N-dimethylaminopyridine or the like.

In embodiments, the process may be carried out at a suitable temperature and for time sufficient for the urea formation, at about 0° C. or above and for at least 1 hour or longer.

In another aspect, the present application provides a process for the preparation of Omecamtiv mecarbil or a salt thereof, comprising the step of reacting methyl 4-(2-fluoro-3-isocyanatobenzyl)piperazine-1-carboxylate with 6-methylpyridin-3-amine.

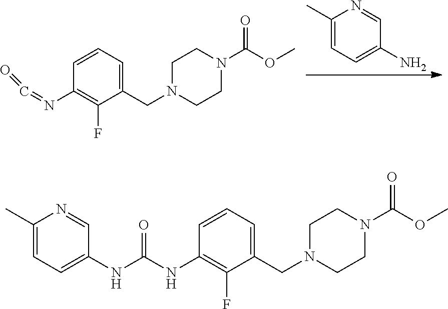

In embodiments, the process of present aspect may be carried out may be carried out in the presence of a suitable solvent. Solvent may be selected from the group consisting of ethers, such as diethyl ether, diisopropyl ether, tetrahydrofuran, MTBE, dioxane, and dimethoxyethane; halogenated hydrocarbons, such as dichloromethane, dichloroethane chloroform, carbon tetrachloride, and chlorobenzene, 1,2-dichlorobenzene; aromatic hydrocarbons, such as toluene, benzene, xylene; aliphatic hydrocarbons such as pentane, hexane, heptane; nitriles such as acetonitrile, esters such as ethyl acetate, isopropyl acetate and polar aprotic solvents such as DMF, DMSO, DMAc, any mixtures of two or more thereof.

In embodiments, the process may be carried out at a suitable temperature and for time sufficient for urea formation, at about 0° C. or above and for at least 1 hour or longer.

In another aspect, the present application provides a process for the preparation of Omecamtiv mecarbil or a salt thereof, comprising the step of reacting methyl 4-(3-cyano-2-fluorobenzyl)piperazine-1-carboxylate with 6-methylpyridin-3-amine in the presence of Trifluoroacetic anhydride (TFAA)/Dimethylsulfoxide (DMSO).

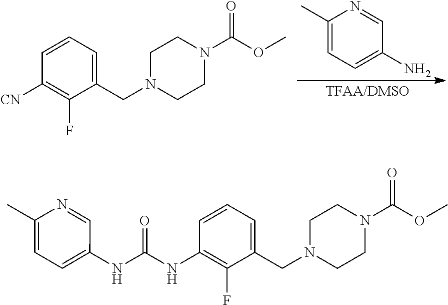

In another aspect, the present application provides Omecamtiv mecarbil or its HCl salt produced according to the aspects of instant application and pharmaceutical compositions thereof, wherein the purity of Omecamtiv mecarbil is more than 95% by HPLC or more than 99% by HPLC. In embodiments, the Omecamtive mecarbil or its HCl salt produced according to the aspects of instant application may be in crystalline or amorphous forms.

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

### Definitions

The term “about” when used in the present application preceding a number and referring to it, is meant to designate any value which lies within the range of ±10%, preferably within a range of 5%, more preferably within a range of 2%, still more preferably within a range of f1% of its value. The term “inert solvent” when used in the present application is a solvent that does not react with the reactants or reagents under conditions that cause the chemical reaction indicated to take place.

### EXAMPLES

### Example-1: Preparation of Methyl piperazine-1-carboxylate

To a mixture of Methyl piperizine-1-carboxylate.HCl (20 g) in dichloromethane (200 mL), 20% aq. potassium hydroxide (140 mL) was added at 30° C. and the reaction mixture was stirred for 1 h at 30° C. The organic dichloromethane layer was separated and aqueous layer was extracted with dichloromethane (2×50 mL). The combined organic layer was washed with brine (200 mL) and dried over sodium sulfate. The solvent was evaporated at 45° C. under reduced pressure to obtain 14.5 g of title compound.

### Example-2: Preparation of phenyl (6-methylpyridin-3-yl)carbamate hydrochloride

The mixture of 5-Amino-2-methyl pyridine (50 g) in acetonitrile (200 mL) was cooled to 20° C. and phenyl chloroformate (60.9 mL) was added to the reaction mixture over a period of 25 minutes and stirred for 3 hour at the same temperature. After the completion of reaction, the precipitated product was filtered and washed with methyl tert-butyl ether and the solid was dried at 40° C. under reduced pressure to obtain 118.0 g of title compound.

### Example-3: Preparation of 3-Amino-2-fluoro benzoic acid

To a mixture of 3-Amino-2-fluoro-benzonitrile (100 g) and water (1 L), sodium hydroxide (29.38 g) was added at 30° C. The reaction mixture was heated to 100° C. and stirred for 14 hours the same temperature. After the completion of reaction, the reaction mixture was cooled to 10° C. and the pH of the reaction mixture was adjusted to 5. The reaction mixture was extracted with ethyl acetate (4×500 mL) and the combined organic layer was washed with brine solution (500 mL). The organic layer was dried over sodium sulfate and the solvent was evaporated under reduced pressure at 45° C. to obtain 83.1 g of title compound as pale yellow solid. HPLC purity: 98.65%

### Example-4: Preparation of methyl 3-Amino-2-fluoro benzoate

A mixture of 3-Amino-2-fluoro-benzoic acid (83 g) and methanol (996 mL) was cooled to 0° C. and thionyl chloride (97 mL) was added to the reaction mixture for 30 minutes at the same temperature. The reaction mixture was heated to 65° C. for 16 hours and cooled to 40° C. Thionyl chloride and excess methanol was evaporated under reduced pressure at 45° C. and the residue was cooled to 10° C. The reaction mixture was quenched with saturated aqueous solution of sodium bicarbonate (1.5 L) to adjust the pH to 7. The reaction mixture was extracted with ethyl acetate (2×500 mL) and the combined organic layer was washed with brine solution (500 mL). The organic layer was dried over sodium sulfate and the solvent was evaporated under reduced pressure at 45° C. to obtain 88.5 g of title compound. HPLC purity: 98.98%

### Example-5: Preparation of methyl 2-fluoro-3-(3-(6-methylpyridin-3-yl)ureido)benzoate

To a mixture of methyl 3-Amino-2-fluoro benzoate (2.0 g) in tetrahydrofuran (20 mL), phenyl (6-methylpyridin-3-yl)carbamate hydrochloride (3.75 g) was added and the reaction mixture was cooled to 0° C. Diisopropyl ethyl amine (6.0 mL) was added to the reaction mixture in 15 minutes and the reaction mixture was heated to 65° C. for 22 hours. The reaction mixture was cooled to 30° C. and quenched with water. The reaction mixture was extracted with ethyl acetate (2×20 mL). The combined organic layer was washed with brine solution (20 mL) and dried over sodium sulfate. The solvent was removed by evaporation under reduced pressure at 45° C. The solid was suspended in diethyl ether (40 mL) at 30° C. for 10 minutes and filtered. Washed the solid with hexane and diethyl ether followed by purification through column (100-200 mesh) chromatography using 60% ethyl acetate in Hexane as eluent to obtain 2.83 g of the title compound as light brown solid. HPLC purity: 90.84%

### Example-5A: Alternate method for the preparation of methyl 2-fluoro-3-(3-(6-methylpyridin-3-yl)ureido)benzoate

To a mixture of methyl 3-Amino-2-fluoro benzoate (0.1 g) in dichloromethane (4 mL), triethylamine (0.33 mL) was added and the reaction mixture was cooled to 5° C. Triphosgene (0.123 mg) was added and the reaction mixture was allowed to warm to 28° C. and stirred at the same temperature for 1 hour. A solution of 5-amino-2-methyl pyridine (0.064 g) in dichloromethane (2 mL) was slowly added at 28° C. and the reaction mixture was stirred at the same temperature for 2 hours. Water (15 mL) was added to the reaction mixture and the precipitated solid was filtered. The wet solid was dried at 40° C. under reduced pressure to obtain the title compound as off-white solid with Mass (m/z): 304.1 [M+H]+

### Example-6: Preparation of 1-(2-fluoro-3-(hydroxymethyl)phenyl)-3-(6-methylpyridin-3-yl)urea

The mixture of methyl 2-fluoro-3-(3-(6-methylpyridin-3-yl)ureido)benzoate (3.3 g), sodium borohydride (2.48 g) and tetrahydrofuran (66 mL) was heated to 65° C. Methanol (19.8 mL) was added to the reaction mixture at 65° C. in 30 minutes and stirred for 2 hours at the same temperature. The reaction mixture was cooled to 30° C. and quenched with saturated ammonium chloride solution (33 mL). The reaction mixture was extracted with ethyl acetate (60 mL). The combined organic layer was washed with brine solution and dried over sodium sulfate. The solvent was evaporated under reduced pressure at 45° C. and the product was purified by column chromatography (100-200 mesh) using 80% ethyl acetate-Hexane as eluent to obtain 2.63 g of the title compound. HPLC purity: 99.35%

### Example-7: Preparation of Omecamtiv Mecarbil

A mixture of 1-(2-fluoro-3-(hydroxymethyl)phenyl)-3-(6-methylpyridin-3-yl)urea (1 g) in dichloromethane (20 mL) was cooled to 0° C. and triethylamine (1.48 mL) was added followed by the addition of methane sulfonylchloride (0.422 mL) into the reaction mixture over a period of 15 minutes. The reaction mixture was warmed to 28° C. and stirred for 3 hours at the same temperature. The reaction mixture was quenched with water (10 mL) and extracted with dichloromethane (2×10 mL). The combined organic layer was washed with brine solution and dried over sodium sulfate. The solvent was evaporated under reduced pressure at 45° C. The product was combined with dimethyl formamide (10 mL) and cooled to 0° C. Potassium carbonate (1.5 g) and potassium iodide (60 mg) and Methyl piperazine-1-carboxylate (786 mg) were added to the reaction mixture. The reaction mixture was allowed to warm to 28° C. and stirred at the same temperature for 16 hours. The reaction mixture was quenched with water (50 mL) and extracted with ethyl acetate (2×50 mL). The combined organic layer was washed with brine solution and dried over sodium sulfate. The solvent was evaporated under reduced pressure at 45° C. and the product was purified by column chromatography (60-120 mesh) using 5-6% methanol-dichloromethane as eluent to obtain 0.988 g of the title compound as pale yellow solid. HPLC purity: 94.53% and PXRD: FIG. 2.

### Example-8: Preparation of 1-(3-cyano-2-fluorophenyl)-3-(6-methylpyridin-3-yl)urea

To a mixture of 3-Amino-2-fluorobenzonitrile (3.5 g) in tetrahydrofuran (40 mL), phenyl (6-methylpyridin-3-yl)carbamate hydrochloride (8.1 g) was added at 30° C. and the reaction mixture was cooled to 0° C. Diisopropyl ethyl amine (6.7 mL) was added slowly and the reaction mixture was heated to 65° C. The reaction mixture was stirred at 65° C. for 22 hours and cold water (50 mL) was added. The reaction mixture was extracted with ethyl acetate (2×50 mL) and the combined organic layer was washed with brine (50 mL) and dried over anhydrous sodium sulfate. The solvent was evaporated under reduced pressure at 45° C. and the product was purified by column (100-200 mesh) chromatography with 10% Methanol-dichloromethane as eluent, to obtain 5.4 g of title compound as yellow solid. HPLC purity: 92.35%

### Example-9: 2-fluoro-3-(3-(6-methylpyridin-3-yl)ureido)benzoic acid

To a mixture of 1-(3-cyano-2-fluorophenyl)-3-(6-methylpyridin-3-yl)urea (2.0 g) in ethanol (10 mL) at 30° C., aqueous sodium hydroxide solution (0.88 g dissolved in 10 mL of water) was added over a period of 25 minutes and the reaction mixture was heated to 100° C. The reaction mixture was stirred for 24 hours at the same temperature and cooled to 0° C. The pH of the reaction mixture was adjusted to 5 using 5N HCl. The precipitated solid was filtered and dried under vacuum to obtain 1.5 g of title compound as pale yellow solid. HPLC purity: 93.79%

### Example-10: Preparation of methyl 2-fluoro-3-(3-(6-methylpyridin-3-yl)ureido)benzoate

A mixture of 2-fluoro-3-(3-(6-methylpyridin-3-yl)ureido)benzoic acid (1.0 g) and methanol (42 mL) was cooled to 0° C. and thionyl chloride (1.25 mL) was added over a period of 10 min. The reaction mixture was heated to 65° C. and stirred at the same temperature for 16 hours. After the completion of reaction excess methanol and thionyl chloride were evaporated under reduced pressure at 45° C. and the residue was cooled to 10° C. and the reaction mixture was quenched with slow addition of saturated sodium bicarbonate solution (20 mL) to adjust the pH to 7. The reaction mixture was stirred for 30 min at 28° C. and extracted with ethyl acetate (2×40 mL). The combined organic layers was washed with brine (40 mL) and dried over sodium sulfate. The solvent was evaporated at 45° C. under reduced pressure to obtain 860 mg of title compound as off-white solid.

### Example-11: Preparation of Omecamtiv Mecarbil

To a mixture of methyl 4-(3-amino-2-fluorobenzyl)piperazine-1-carboxylate (50 mg) and dichloromethane (2 mL), triethylamine (75 mg) was added at 28° C. The reaction mixture was cooled to 5° C. and triphosgene (39 mg) was added at the same temperature. The reaction mixture was allowed to warm to 28° C. and stirred at the same temperature for 1 hour. A solution of 5-Amino-2-methyl pyridine (20.2 mg) in dichloromethane (1.5 mL) was added at 28° C. and stirred the reaction mixture at same temperature for 2 hours. The reaction mixture was quenched with water (15 mL) and extracted with dichloromethane (3×10 mL). The combined organic layer was dried over anhydrous sodium sulfate and the solvent was evaporated at 40° C. under reduced pressure to obtain the title compound, confirmed, by Mass (M+H)⁺: 402.1.

### Example-12: Preparation of Omecamtiv Mecarbil

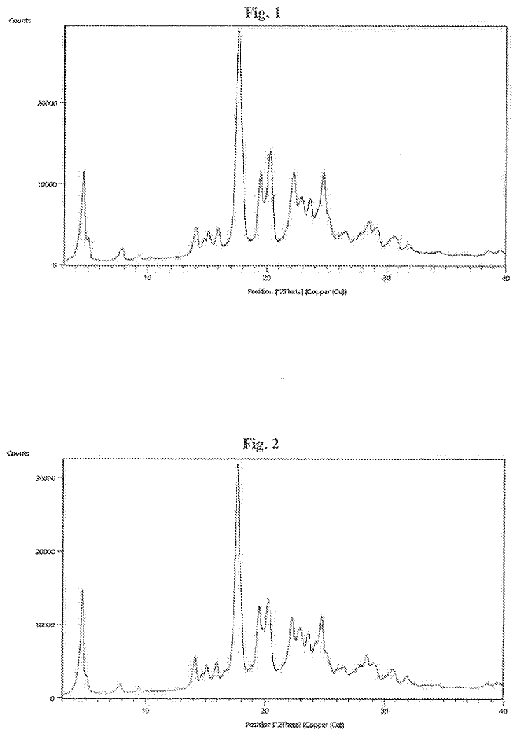

To a mixture of methyl 4-(3-amino-2-fluorobenzyl)piperazine-1-carboxylate (52 g), tetrahydrofuran (130 mL) and acetonitrile (130 mL) at 28° C., phenyl (6-methylpyridin-3-yl)carbamate hydrochloride (51.5 g) and then diisopropylethylamine (40.6 mL) were added and the reaction mixture was heated to 55° C. The reaction mixture was stirred at the same temperature for 18 hours and water (780 mL) was added slowly. The reaction mixture was cooled to 20° C. and stirred at the same temperature for 30 minutes. The solid was filtered and washed with water (156 mL) and acetonitrile (2×156 mL). The solid was dried at 45° C. under reduced pressure and purified by silica gel (60-120 mesh) column chromatography using 5-6% MeOH/dichloromethane as eluent. Fractions containing the product were concentrated under reduced pressure at 45° C. to obtain 47 g of the title compound as off-white solid. Purity by HPLC: 99.67% & PXRD: FIG. 1.

### Example-13: Preparation of Dihydrochloride Salt of Omecamtiv Mecarbil

A mixture of Omecamtiv mecarbil (68 g), isopropyl alcohol (176.8 mL) and water (102 mL) was heated to 45° C. and 6 M HCl (62 mL) was added slowly. The reaction mixture was then heated to 60° C. and stirred at the same temperature for 30 minutes. The reaction mixture was cooled to 45° C. and stirred at the same temperature for 1 hour. Isopropyl alcohol (251.6 mL) was added slowly at 45° C. and the reaction mixture was heated to 55° C. The reaction mixture was stirred at the same temperature for 1 hour and then cooled to 45° C. The reaction mixture was stirred further for 2 hours at 45° C. and isopropyl alcohol (496.4 mL) was added slowly. The reaction mixture was stirred at the same temperature for 1 hour and cooled to 20° C. The reaction mixture was stirred at the same temperature for 12 hours and the solid was filtered. The solid was washed with 95:5 isopropyl alcohol and water (68 mL) and isopropyl alcohol (136 mL) and dried under reduced pressure at 45° C. for 30 minutes to obtain 67.6 g of the title compound as off-white solid. Purity by HPLC: 99.94%

1. A process for the preparation of Omecamtiv mecarbil or a salt thereof, comprising a step of converting a urea compound of formula-II: 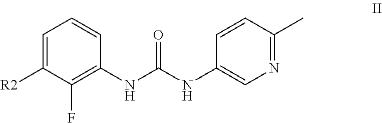
to Omecamtiv mecarbil, wherein R₂ is selected from the group consisting of cyano, formyl, carboxylic acid or ester thereof, hydroxymethyl, sulfonyloxy methyl and halomethyl.

2. The process of claim 1, wherein the urea compound of formula II is converted to Omecamtiv mecarbil by reacting with a piperazine compound of formula-III, wherein P is a nitrogen protecting group or CO₂CH₃

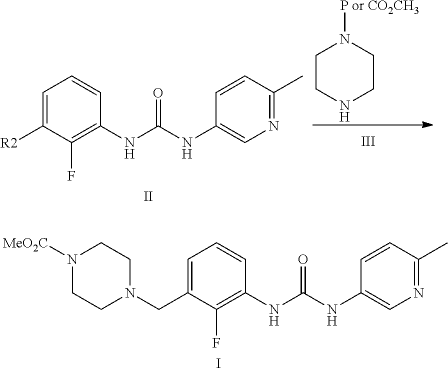

3. An urea compound of formula-II, 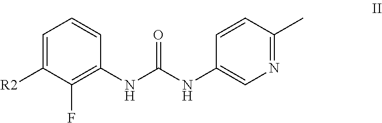
wherein R₂ is selected from the group consisting of cyano, formyl, carboxylic acid or ester thereof, hydroxymethyl, sulfonyloxy methyl and halo methyl.

4. A process for the preparation of an urea compound of formula-II as defined in claim 3, comprising the step of reacting an aniline compound of formula-IV, 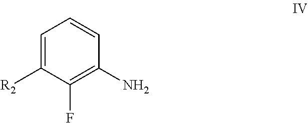
or its derivative, wherein R₂ is selected from the group consisting of cyano, formyl carboxylic acid or ester thereof, hydroxymethyl, sulfonyloxy methyl and halo methyl with a 6-methylpyridin-3-amine, or its derivative, optionally in the presence of carbonyl source.

5. The process of claim 4, wherein the derivative is selected from isocyanate or N-acylated derivatives.

6. The process of claim 5, wherein the N-acylated derivative of 6-methylpyridin-3-amine is selected from derivatized compounds of formula IVa 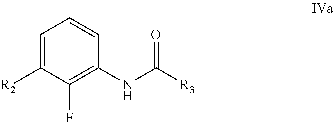
or formula V, 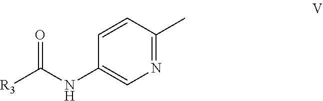
wherein R₃ is selected from the group consisting of halogen, alkoxy, aryloxy and heteroaryl.

7. A process for the preparation of Omecamtiv mecarbil or a salt thereof, comprising the step of reacting a derivatized aniline of formula VI 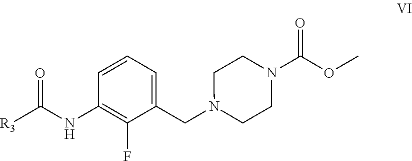
with 6-methylpyridin-3-amine, wherein R₃ is selected from the group consisting of halogen, alkoxy, aryloxy and heteroaryl.

8. A process for the preparation of Omecamtiv mecarbil or a salt thereof, comprising the step of reacting methyl 4-(3-amino-2-fluorobenzyl)piperazine-1-carboxylate with 6-methylpyridin-3-amine in the presence of a carbonyl source.

9. A process for the preparation of Omecamtiv mecarbil or a salt thereof, comprising the step of reacting methyl 4-(2-fluoro-3-isocyanatobenzyl)piperazine-1-carboxylate with 6-methylpyridin-3-amine.

10. The process of claim 4, wherein the carbonyl source is selected from the group consisting of carbon monoxide, phosgene, diphosgene, triphosgene, carbonyldiimidazole (CDI), 1,1-carbonylbisbenzotriazole, dimethyl carbonate diethyl carbonate, diisopropyl carbonate, S,S-Dimethyldithiocarbonate (DMDTC), di-tert-butyl dicarbonate, bis(4-nitrophenyl)carbonate, diisopropyl carbodimide, dicyclohexyl carbodiimide and phenyl chloroformate.

11. The process of claim 8, wherein the carbonyl source is selected from the group consisting of carbon monoxide, phosgene, diphosgene, triphosgene, carbonyldiimidazole (CDI), 1,1-carbonylbisbenzotriazole, dimethyl carbonate diethyl carbonate, diisopropyl carbonate, S,S-dimethyldithiocarbonate (DMDTC), di-tert-butyl dicarbonate, bis(4-nitrophenyl)carbonate, diisopropyl carbodimide, dicyclohexyl carbodiimide and phenyl chloroformate.
